(19) United States
(12) Patent Application Publication
(10) Pub. No.: US 2003/0078567 A1
(43) Pub. Date: Apr. 24, 2003

(54) Method and apparatus for laser ThermoProtectiveTreatment(TPT) with pre-programmed variable irradiance long exposures

(76) Inventors: Giorgio Dorin, Cupertino, CA (US); Robert P. Murphy, Ellicot city, MD (US); David M. Buzawa, San Jose, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/260,228

(22) Filed: Sep. 27, 2002

Related U.S. Application Data
(63) Continuation-in-part of application No. 09/844,445, filed on Apr. 27, 2001.
(60) Provisional application No. 60/325,895, filed on Sep. 27, 2001.

(51) Int. Cl. A61B 18/20

(52) U.S. Cl. 606/4; 606/6; 606/12

(57) ABSTRACT

An electro-optical system is provided for use with a target site and configured with pre-programmable energy output functions for customized TPT protocols. One or more laser sources are provided producing an output beam. A control device is coupled to the laser source. The control device includes a memory that stores at least one laser source energy output function. The laser source energy output function is used to create an intra-operatively invisible therapeutic treatment with enhanced thermotolerance which minimize iatrogenic damage to surrounding structures.

### CROSS-REFERENCE TO RELATED APPLICATIONS

This application claims the benefit of U.S. Serial No. 60/325,895, filed Sep. 27, 2001, and is also a continuation-in-part of U.S. Ser. No. 09/844,445, filed Apr. 27, 2001, both of which are incorporated herein by reference.

### BACKGROUND OF THE INVENTION

1. Field of the Invention

This invention relates generally to an apparatus, and its methods of use, for delivering an intra-operatively invisible therapeutic treatment at a target site, using a novel TPT protocol, which simultaneously, with the treatment, promotes natural thermo-protective reactive mechanisms and enhances thermotolerance to minimize iatrogenic damage to adjacent non-target sites.

2. Description of The Related Art

Lasers are currently used for the treatment of various pathologies of the eye, such as retinal disorders and glaucoma. Glaucoma disorders treatable with laser include open angle glaucoma, angle closure glaucoma and neovascular-refractory glaucoma. Retinal disorders treatable with laser include diabetic retinopathy, macular edema, central serous retinopathy, age-related macular degeneration (AMD), and the like.

Diabetic retinopathy represents the major cause of severe vision loss (SVL) for people up to 65 years of age, while AMD represents the major cause of SVL in people between 65 and 80 years of age. More than 32,000 Americans are blinded from diabetic retinopathy alone, with an estimated 300,000 diabetics at risk of becoming blind. The incidence of AMD in the USA is currently estimated at 2 million new cases per year, including 1.8 million cases with the “dry” form and 200,000 cases with the “wet” form or choroidal neovascularization (CNV). The most widely used form of laser treatment for ocular disorders is called laser photocoagulation (P.C.).

Laser P.C. has become the standard treatment for a number of retinal disorders such as macular edema, central serous retinopathy, proliferative diabetic retinopathy, CNV, and the like. Laser P.C. is a photo-thermal process in which the laser energy is directed and absorbed by endogenous chromophores and converted into heat. This localized thermal elevation causes a therapeutic “damage” which can span from protein denaturation to coagulation necrosis. This photo-thermal damage has the purpose to induce physiological healing responses that mediate biological chain of events, leading to the therapeutic benefits of laser P.C.

More recently, lasers have been also used for the photo-activation of exogenous photosensitizing drugs to produce localized photo-chemical therapeutic damages as in, but not restricted to, photo dynamic therapy (PDT).

The human neuro-sensory retina is basically transparent and does not interact with most of the wavelengths emitted by ophthalmic lasers systems currently in use. Thus a laser photo-thermal event cannot originate in the neuro-sensory retina, and the laser irradiance levels used in conventional retinal P.C. protocols do not cause direct damage to its structure.

Conventional retinal P.C. relies on some visible “blanching” of the retina as the treatment endpoint. Since the laser energy does not damage directly the neuro-sensory retina, the visible “blanching” endpoint is the sign that its normally transparent structure starts scattering light because it has been indirectly damaged by the conduction of a thermal wave caused by a thermal elevation originated elsewhere. Normally the thermal elevation originates underneath the neuro-sensory retina, where natural chromophores (i.e. melanin) contained in the retinal pigment epithelium (RPE) and in choroidal melanocytes absorb the laser energy.

A visible retinal “blanching” is a convenient and practical end-point for the surgeon, but it also constitutes a iatrogenic damage to the neurosensory retina with undesirable adverse complications including some vision loss, decreased contrast sensitivity and reduced visual fields in a substantial number of patients.

The thermal elevation can be controlled by (i) laser irradiance (power density), (ii) exposure time and (iii) wavelength. High thermal elevations are normally created with current clinical protocols that are aimed to produce visible endpoints ranging from intense retinal whitening (full thickness retinal burn) to barely visible retinal changes. Although laser P.C. has been proven therapeutically effective and constitutes the standard-of-care in preventing severe vision loss (SVL) in various ocular disorders, it is now recognized that any visible endpoint with retina blanching is by definition the result of an excessive thermal elevation at the RPE, often associated with irreversible changes of the RPE and with localized area of geographic atrophy, which may be unnecessary and should be avoided.

A geographic atrophy represents an irreversible scotoma (blind spot) and the change of the RPE optical characteristics constitute the loss of the “absorbing chromophores” that mediate the conversion of laser energy into heat, a loss which prevents any further laser treatment in that area.

Accordingly, there is a need for an apparatus and method that avoids or minimize unnecessary iatrogenic damage to the neuro-sensory retina in laser procedures. There is a further need for an apparatus, and its methods of use, that minimize unnecessary iatrogenic damage by confining the thermal elevation at the intended target tissue and by stimulating natural thermo protective mechanisms capable of increasing the thermotolerance of adjacent non-target tissue.

### SUMMARY OF THE INVENTION

Accordingly, an object of the present invention is to provide a treatment apparatus, and its methods of use, for performing laser ThermoProtectiveTreatment (TPT) procedures with minimum possible damage to surrounding non-intended targets.

Another object of the present invention is to provide a treatment apparatus, and its methods of use, that produces neuroretina-sparing therapeutic photothermal and/or photochemical effects.

A further object of the present invention is to provide a treatment apparatus, and its methods of use, that uses programmed variable irradiance prolonged laser exposure, which does enhance the thermotolerance while the treatment is in progress.

Still another object of the present invention is to provide a treatment apparatus, and its methods of use, that allows a therapeutic treatment, which is so delicate that is intra-operatively unperceivable by the patient and invisible to the operator (lack of treatment visibility implies no damage to neuro sensory retina tissue).

Another object of the present invention is to provide a treatment apparatus, and its methods of use, which is programmable to work with feedback signals proportional to intra-operative changes of physical or physiological parameters, thus with real-time detection and monitoring of treatment-induced sub-clinical (invisible) effects at the target site.

These and other objects of the present invention are achieved in an electro-optical system for use with a target site and configured with pre-programmable energy output functions for customized TPT protocols. One or more laser sources are provided producing an output beam. A control device is coupled to the laser source. The control device includes a memory that stores at least one laser source energy output function. The laser source energy output function is used to create an intra-operatively invisible therapeutic treatment with enhanced thermotolerance which minimize latrogenic damage to surrounding structures.

In another embodiment of the present invention, an electro-optical system is provided for use with a target tissue. One or more laser sources are included for producing an output beam. A programmable control device is coupled to the one or more laser sources. The control device includes a memory that stores at least one laser source energy output functions. A delivery device is coupled to the one or more laser sources. The delivery device delivers at least a portion of the output beam to the target tissue. A monitoring device is coupled to at least one of the control device or to an operator's device.

In another embodiment of the present invention, a method for treating a target tissue provides an apparatus that produces pre-programmed energy output functions. A treatment beam is delivered that utilizes at least one of the pre-programmed energy output functions to stimulate natural thermoprotective reactive mechanisms while performing an intra-operatively invisible therapeutic treatment at the target tissue.

In another embodiment of the present invention, a method for treating a target tissue provides one or more laser sources coupled to a control device. The control device has a memory that stores at least one laser source energy output function. A treatment beam is delivered from the one or more laser sources to the target tissue. Natural thermoprotective mechanisms are stimulated while creating an intra-operatively invisible thermo protected therapeutic treatment of the target tissue.

### BRIEF DESCRIPTION OF THE DRAWINGS

FIG. 1 depicts an embodiment of the laser apparatus of the present invention.

FIGS. 2a-2c depict an example of a typical TPT variable irradiance laser output function of the present invention.

### DETAILED DESCRIPTION

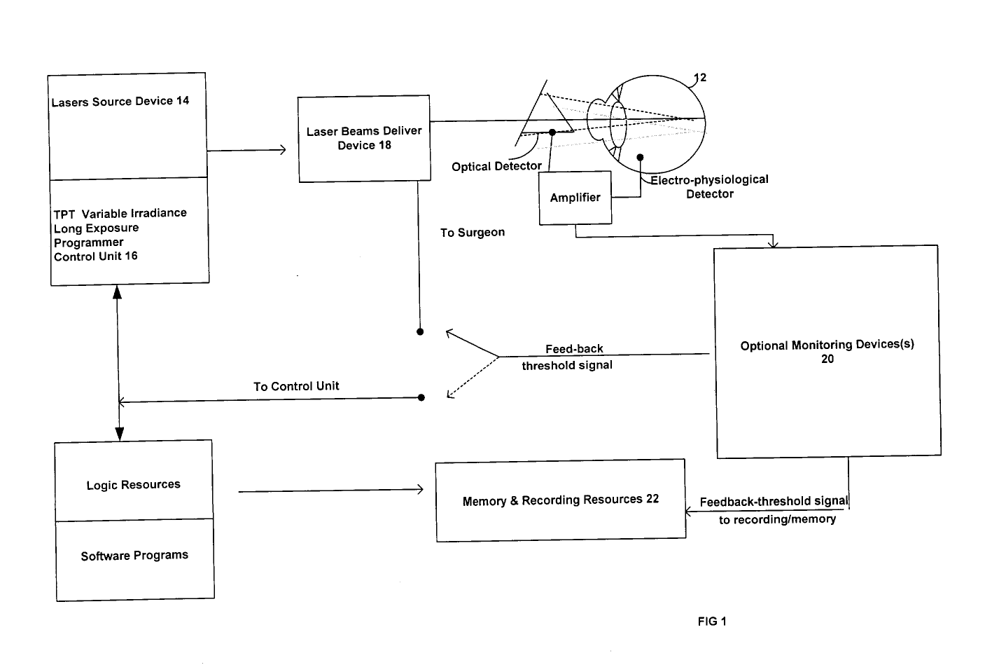

Referring to FIG. 1, one embodiment of the present invention is a TPT ophthalmic laser apparatus 10. Apparatus 10 can be configured to produce neuroretina-sparing therapeutic photothermal and/or photochemical effects as a result of programmed variable irradiance prolonged laser exposure incident to targeted tissue site 12, including but not limited to an ocular tissue site. Apparatus 10 can include one or more optical source devices 14 including but not limited to a laser. Devices 14 can include one or more lasers that can operate at various wavelengths and whose emission is controlled by a ThermoProtectiveTreatment (TPT) variable irradiance, exposure, pulse regime programmer/control unit device (hereafter control device 16) that is delivered through a laser delivery system device 18.

Laser delivery system device 18 can be a variety of different devices including but not limited to a slit lamp delivery system, and the like. In one embodiment, device 14 can be one or more lasers known in the art including but not limited to, ion, dye lasers, Nd:YAG, frequency-doubled Nd:YAG, visible and invisible diode, infrared lasers, and the like. The output of device 14 can include various aiming and treatment beams with the same or different wavelengths. In other embodiments, device 14 can be a photocoagulation source device including but are not limited to infrared lamps, flash lamps, mercury vapor lamps, and the like.

Optionally, one or more monitoring devices 20, capable of detecting treatment-induced sub-clinical changes can be provided. Examples of different monitoring devices 20 include but are not limited to devices that are, optical (interferometry, reflectometry, light scattering, fluorescence or IR imaging), thermometric, electro-physiologic (focal ERG), and the like. Monitoring device 20 can provide feedback signals of intra-operative sub-clinical changes and thresholds (i.e. minimum therapeutic damage MTD, and maximum functional damage MFD, etc.) to the surgeon through an audio/visual device and/or optionally, to control device 16.

Apparatus 10 allows the surgeon to create and select prolonged step-by-step sequentially variable laser irradiation programs. Each program can deliver energy from one or more devices, such as lasers 14 with, (i) various spot sizes and patterns and (ii) various series of subsequent trains of repetitive laser pulses. The output of apparatus 10 can be programmed or changeable, for a sequence of changes, relative to, (i) wavelength, (ii) power, (iii) irradiance, (iv) duty cycle, (v) repetition rate, (vi) exposure time, (vii) repetition interval and the like. These sequence of changes constitute the energy output function, which can be individually set and programmed for the entire duration of the laser treatment, in accordance with various protocols (focal, grid, diffuse with large spot, and the like) requested for addressing the therapeutic needs of specific disorders.

With apparatus 10, the treatment is normally under the surgeon's control. Optionally the treatment can be assisted and/or controlled by feed-back signals from the real time monitoring of the intra-operative induced changes of physical (thermal, optical, etc.) or physiological (ERG, autofluorescence, etc.) parameters. Such feed-back signals can be used for, (i) providing the real-time detection of the sub-clinical (invisible) therapeutic treatment window, above the a) minimum therapeutic damage (MTD) threshold and not exceeding the b) maximum functional damage (MFD) threshold; (ii) providing perceptible signals (i.e. audio or others) to the physician as well as electric signals for the optional automatic control of the emission of the TPT ophthalmic laser apparatus, (iii) the recording of all successfully delivered MTD applications, their location in the ocular fundus and other relevant data pertaining to the treatment, and the like.

Apparatus 10, and its methods of use, provide a practical solution to the challenges and difficulties posed by minimum intensity photocoagulation (MIP) sub-clinical treatments of ocular pathologies requiring prolonged exposure with the minimal possible retinal damage and related iatrogenic vision impairment.

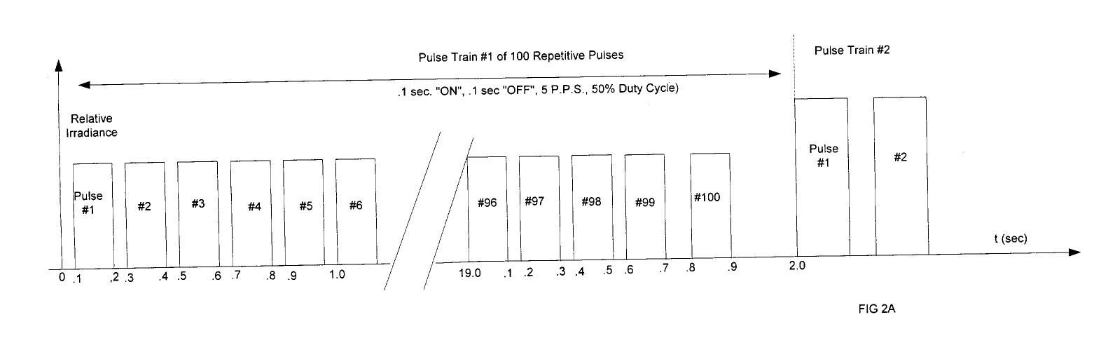

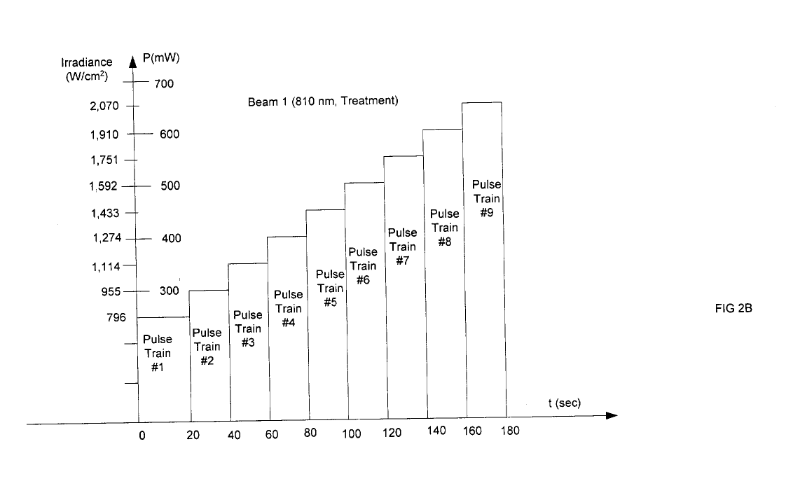

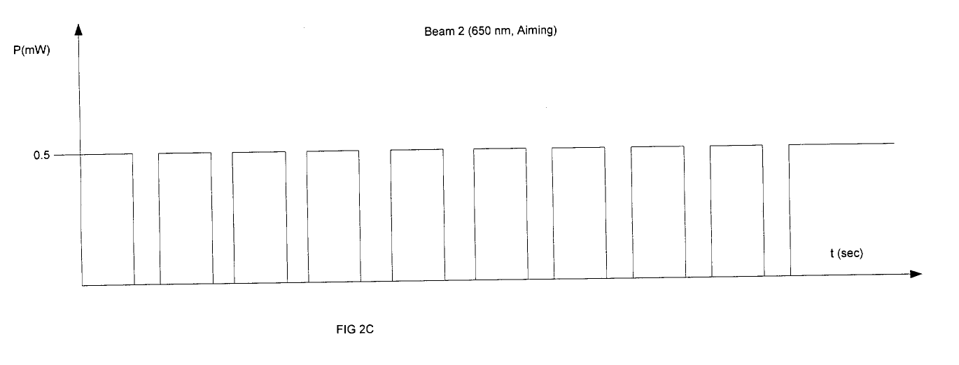

The programming capabilities of apparatus 10, particularly with control device 16, are broad reaching. A simple example is represented by the irradiance histogram in FIGS. 2a-c, showing a pre-programmed laser energy output function intended for, but not limited to, the occlusion of subretinal CNVs' feeder vessels. This particular pre-programmed laser energy output function is designed to allow the closure of a deep vascular structure, naturally thermally protected by blood flow, through a prolonged thermal elevation (time-temperature-history) eventually causing vascular thrombosis, sclerosis or leukostasis, while auto regulating intraoperatory and temporary bio-physical changes, which allow deeper penetration and lower thermal elevation without visible permanent changes of RPE optical properties, nor scars or geographic atrophy.

Control device 16 can provide a variety of pre-programmed laser energy output functions, in the form of software programs, databases and the like that can be stored in one or more memories 22. Memory 22 is configured to store laser energy output functions programs, data, data sets and databases, including but not limited to achieved MTD thresholds that can be confirmed by monitoring device 20. Examples of programs include control algorithms such as proportional, proportional derivative and proportional derivative integral (PID) algorithms. Suitable memories 22 include but are not limited to, RAM, ROM, PROM, flash memory and the like. Suitable data and databases that can be stored include but are not limited to, optical interference patterns and profiles data, other optical data and the like. A database of such information can be both for a population or an individual patient and may include baseline (e.g. pretreatment), treatment and post-treatment profiles.

In various embodiments, apparatus 10 is an ophthalmic apparatus, and its methods of use, for delivering TPT for retina-sparing subthreshold minimum intensity photocoagulation (MIP). Apparatus 10 achieves this while minimizing iatrogenic damage. TPT is a variable-irradiance long exposure protocol in which natural thermo protective mechanisms are stimulated during the delivery of the therapeutic treatment.

In one embodiment, apparatus 10 is a TPT ophthalmic laser apparatus with irradiance that can be time-step-programmed. The time-step-preprogrammed creates localized time-temperature-histories and produces photothermal and/or photochemical therapeutic effects. This can be achieved while simultaneously inducing biochemical and biophysical changes which increase the thermotolerance and thermoresistance, the temporary state of resistance to heat killing of the retina. TPT allows for completion of an intended therapeutic tasks with reduced thermal hazards and with minimal or no signs of damage visible during the treatment.

More specifically, apparatus 10 can include an ophthalmic laser device 14, which can include one or more laser sources. All or a portion of the laser parameters, including but not limited to power, irradiance, pulse “ON” time, inter-pulse “OFF” time, exposure duration, number of pulses, repetition interval and the like, can be set individually in order to create a variety of pre-programmed laser energy output functions. Each output function's program can be designed to gradually produce the intended photothermal and/or photochemical therapeutic effects while simultaneously, (i) modulating natural protective mechanisms capable of increasing the thermal tolerance of the retina (vascular auto-thermo-regulation, upregulation of neuroprotective agents, synthesis of heat shock proteins, and the like) and (ii) altering biophysical and bio-chemical properties of endogenous and exogenous laser absorbing chromophores, for effectively sparing the sensory retina while addressing the targeted sub retinal structures, whose treatment can benefit from prolonged exposures.

The combined processes of, (i) photo-thermal and/or photo-chemical sub-retinal therapeutic damage and (ii) photo-thermal inner-retina protection and conditioning, can be simultaneously or sequentially accomplished with laser energy output functions that are programmed to deliver the laser energy in series of subsequent trains of laser pulses. The output of laser devices 14 has parameters, including but not limited to, wavelength, power, irradiance, duty cycle, repetition rate, repetition interval, exposure time, and the like, can be individually programmed with control device 16 for the entire duration of the laser treatment. Furthermore, intra-operative monitoring of induced changes of physical (thermal, optical, and the like) or physiological (ERG, autofluorescence, etc.) parameters can provide feed-back signals. Such feedback signals can assist the surgeon in the completion of the sub-clinical (invisible) treatment or, alternatively, utilized for the automatic control of laser devices 14. Intra-operative physical and/or physiological monitoring techniques to assist subclinical threshold treatments include, but are not limited to, the use of thermometry, reflectometry, interferometric reflectometry, light scattering, focal electroretinography, fluorescence, autofluorescence imaging, SLO imaging, and the like.

With apparatus 10, and its methods of use, minimally invasive TPT protocols can be performed where, (i) the therapeutic application can be administered in such a way to effectively treat the target and while changing (ii) biochemical and biophysical properties that enhance the thermal protection and/or thermotolerance of overlying non-targets. Apparatus 10, and its methods of use, are particularly useful for minimally invasive and clinically effective treatments of selected tissue and/or structures within the eye, without the need for visible endpoints, while minimizing injury, such as thermal injury, to surrounding structures including the neurosensory retina. It will be appreciated, that apparatus 10, and its methods of use, can be utilized with any tissue target.

While the invention has been described in connection with what is presently considered to be the most practical and preferred embodiment, it is to be understood that the invention is not limited to the disclosed embodiment, but on the contrary it is intended to cover various modifications and equivalent arrangement included within the spirit and scope of the claims which follow.

What is claimed is:

1. An electro-optical system for use with a target tissue, configured with pre-programmable energy output functions for customized TPT protocols, comprising:
one or more laser sources producing an output beam; and
a control device coupled to the one or more laser sources, the control device including a memory that stores at least one laser source energy output function used to create an intra-operatively invisible therapeutic treatment with enhanced thermotolerance which minimizes iatrogenic damage to structures near the target tissue.

2. The system of claim 1, wherein the target tissue is selected from at least one of an ocular structure, a tumor, and an epidermal tissue.

3. The system of claim 2, wherein the oculator structure is selected from sub-retinal afferent or efferent vessels feeding choroidal neovascular membranes, retinal angiomatous proliferation (RAP), retinal pigment epithelium (RPE), and choroidal neovascular membranes (CNVM).

4. The system of claim 1, further comprising:
a monitoring device coupled to the control device, the monitoring device configured to receive a target tissue parameter from the target tissue to provide a feedback signal to at least one of the operator of the one or more laser sources or the control device, and provide a real-time detection of a therapeutic treatment window for the target tissue that is invisible to an operator of the one or more laser sources.

5. The system of claim 4, wherein the one or more laser source energy output function is selected from at least one of wavelength, power, irradiance, duty cycle, repetition rate, exposure time cycle, repetition rate and exposure time.

6. The system of claim 4, wherein the target tissue parameter is at least one of optical, thermometric and electro-physiologic parameter to produce the feedback signal to the control device.

7. The system of claim 6, wherein the optical parameter is selected from at least one of interferometry, reflectometry, fluorescence and IR imaging.

8. The system of claim 4, wherein the feedback signal is used to provide delivery of the output beam with different spot size.

9. The system of claim 4, wherein the feedback signal is used to provide delivery of the output beam with different patterns.

10. The system of claim 4, wherein the feedback signal is sued to provide a setting of the one or more laser source energy output function.

11. The system of claim 1, further comprising:
a delivery device coupled to the one or more laser sources.

12. The system of claim 11, wherein the delivery device includes a slit lamp.

13. The system of claim 1, wherein the one or more laser sources produces a treatment beam and an aiming beam.

14. The system of claim 13, wherein the treatment beam and the aiming beam have different wavelengths.

15. The system of claim 13, wherein the treatment beam and the aiming beam have the same wavelengths.

16. The system of claim 1, wherein the memory is selected from at least one of a RAM, ROM, PROM, EPROM and flash memory.

17. An electro-optical system for use with a target tissue, comprising:
one or more laser sources producing an output beam;
a programmable control device coupled to the one or more laser sources, the control device including a memory that stores at least one laser source energy output function;
a delivery device coupled to the one or more laser sources, the delivery device delivering at least a portion of the output beam to the target tissue; and
a monitoring device coupled to at least one of the control device or to an operator's device.

18. The system of claim 17, wherein the monitoring device provides a feedback signal responsive to intra-operative changes of physical or physiological parameters at the target tissue.

19. The system of claim 18, wherein the monitoring device provides real-time monitoring of treatment-induced invisible effects at the target tissue.

20. The system of claim 17, wherein the target tissue is a sub-retinal structure tissue with sub-retinal CNVs' feeder vessels.

21. The apparatus of claim 18, wherein in response to the feedback signal an irradiance exposure of the output beam to the target tissue is modified.

22. The system of claim 17, wherein the at least one laser source energy output function is selected from at least one of wavelength, power, irradiance, duty cycle, repetition rate, exposure time cycle, repetition rate and exposure time.

23. The system of claim 18, wherein a target tissue parameter is used to produce the feedback signal.

24. The system of claim 23, wherein the target tissue parameter is at least one of optical, thermometric and electro-physiologic.

25. The system of claim 23, wherein the optical parameter is selected from at least one of interferometry, reflectometry, fluorescence and IR imaging.

26. The system of claim 18, wherein the feedback signal is used to provide delivery of the output beam with different spot size.

27. The system of claim 18, wherein the feedback signal is used to provide delivery of the output beam with different patterns.

28. The system of claim 18, wherein the feedback signal is used to provide a setting of the at least one laser source energy output function.

29. The system of claim 17, wherein the laser source produces a treatment beam and an aiming beam.

30. The system of claim 29, wherein the treatment beam and the aiming beam have different wavelengths.

31. The system of claim 29, wherein the treatment beam and the aiming beam have the same wavelengths.

32. The system of claim 17, wherein the memory is selected from at least one of a RAM, ROM, PROM, EPROM and flash memory.

33. A method for treating a target tissue, comprising:
providing an apparatus that produces pre-programmed energy output functions; and
delivering an intra-operatively invisible therapeutic treatment beam utilizing at least one of the pre-programmed energy output functions to stimulate natural thermoprotective reactive mechanisms at the target tissue.

34. The method of claim 33, wherein the natural thermoprotective reactive mechanism is selected from at least one of increased blood flow, swelling, bleaching of endogenous chromophores, and expression of heat shock proteins.

35. The method of claim 33, wherein the target tissue is sub-retinal structure tissue.

36. The method of claim 34, wherein the sub-retinal structure tissue includes sub-retinal CNVs' feeder vessels.

37. The method of claim 33, further comprising:
monitoring the therapeutic treatment at the target tissue.

38. The method of claim 37, wherein the apparatus includes a laser source and a control device.

39. The method of claim 38, further comprising:
in response to monitoring the therapeutic treatment, receiving a target tissue parameter from the target tissue; and
in response to receipt of the target tissue parameter providing a feedback signal to at least one of the operator of the laser source or the control device

40. The method of claim 39, further comprising:
in response to the feedback signal, provide a real-time detection of a therapeutic treatment window for the target tissue that is invisible to the operator of the optical system.

41. A method for treating a target tissue, comprising:
providing one or more laser sources coupled to a control device, the control device including a memory that stores at least one laser source energy output function;
delivering a treatment beam from the one or more laser sources to the target tissue; and
stimulating natural thermoprotective mechanisms while creating an intraoperatively invisible thermo protected therapeutic treatment of the target tissue.

42. The method of claim 41, wherein the target tissue is a sub-retinal structure and the intra-operatively invisible thermo protected therapeutic treatment is stimulated while minimizing damage to a neuro sensory retina tissue.

43. The method of claim 41, wherein the at least one laser parameter is used to assist in creating the therapeutic treatment of the sub-retinal structure tissue.

44. The method of claim 43, wherein the sub-retinal structure tissue includes sub-retinal CNVs' feeder vessels.

45. The method of claim 41, further comprising:
monitoring the intra-operatively invisible thermo protected therapeutic treatment.

46. The method of claim 45, further comprising:
in response to monitoring the intra-operatively invisible thermo protected therapeutic treatment, receiving a target tissue parameter from the target tissue; and
in response to receipt of the target tissue parameter, providing a feedback signal to at least one of the operator of the laser source or the control device.

47. The method of claim 46, further comprising:
in response to the feedback signal, provide a real-time detection of a therapeutic treatment window for the target tissue.

48. The method of claim 41, wherein the at least one laser source parameter is selected from at least one of wavelength, power, irradiance, duty cycle, repetition rate, exposure time cycle, repetition rate and exposure time.

49. The method of claim 46, wherein the target tissue parameter is at least one of optical, thermometric and electro-physiologic parameter to produce the feedback signal to the control device.

50. The method of claim 49, wherein the optical parameter is selected from at least one of interferometry, reflectometry, fluorescence and IR imaging.
